(19) United States
(12) Patent Application Publication
(10) Pub. No.: US 2011/0086102 A1
(43) Pub. Date: Apr. 14, 2011

(54) DELAYED RELEASE COMPOSITIONS

(75) Inventors: David Isaac SILVER, Netanya (IL); Julia Hrakovsky, Rosh Ha-Ayin (IL); Roey Solomonovich, Tel Aviv (IL); Dafna Arieli, Kadima (IL)

(73) Assignee: Teva Pharmaceutical Industries Ltd., Petah Tiqva (IL)

(21) Appl. No.: 12/578,434

(22) Filed: Oct. 13, 2009

(51) Int. Cl. A61K 9/00; A61K 31/343; A61P 37/06

(52) U.S. Cl. 424/487; 514/470; 424/484; 424/488; 424/486

(57) ABSTRACT

The present invention provides delayed release pharmaceutical compositions comprising an active pharmaceutical ingredient, e.g. mycophenolate sodium, and an enteric polymer, and methods for preparing the same. Preferably, the pharmaceutical compositions do not contain a coating.

### FIELD OF THE INVENTION

The invention encompasses delayed release pharmaceutical compositions comprising mycophenolic acid, a pharmaceutically acceptable salt thereof or combinations thereof, and methods for making the same.

### BACKGROUND OF THE INVENTION

Mycophenolic acid was first isolated in the 19th century and has reported activity as an anti-tumor, anti-viral, immunosuppressive, anti-psoriatic, and anti-inflammatory agent.

In the 1960's, Eli Lilly reported the inhibiting effect of mycophenolate sodium salt on the growth of malignant tumor cells in warm-blooded mammals. Mycophenolic acid can be formed either as mono- or disodium mycophenolate. South African Patent No. 6804959 describes the preparation of mono-sodium or disodium mycophenolate.

As the sodium salt, mycophenolate sodium is chemically named (E)-6-(4-hydroxy-6-methoxy-7-methyl-3-oxo-1,3-dihydro-isobenzofuran-5-yl)-4-methylhex-4-enoic acid sodium salt. Its empirical formula is C₁₇H₁₉O₆Na. The molecular weight is 342.32 and the structural formula is:

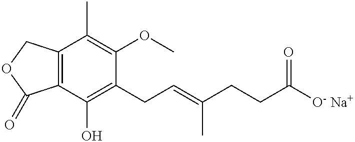

Mycophenolate sodium is a white to off-white, crystalline powder and is highly soluble in aqueous media at physiological pH and practically insoluble in 0.1N hydrochloric acid.

As a consequence of the above solubility properties exhibited by mycophenolate sodium, it had been necessary to develop a mycophenolate composition having a dissolution profile that takes account of the effect pH has on its solubility. In particular, the pH of the stomach is acidic (pH ˜3), and therefore mycophenolate sodium has a tendency to precipitate in the stomach. This precipitation adversely affects the bioavailability of the active ingredient. It is therefore desirable that dissolution of mycophenolate compositions is prevented in the stomach and instead dissolution is delayed until the intestines in which the pH is more basic (pH ˜5.5). Consequently, it has been necessary to develop pharmaceutical compositions comprising a mycophenolate salt that prevents release of the active ingredient in the stomach and instead releases the mycophenolate salt in the upper tract of the intestines.

U.S. Pat. No. 6,025,391 describes pharmaceutical compositions which have been modified to release pharmaceutically acceptable mycophenolate salt in the upper part of the intestinal tract and methods of treatment using the pharmaceutical compositions. The patent discloses a pharmaceutical composition comprising an enteric coated pharmaceutically acceptable mycophenolate salt. An enteric coated capsule containing particles of mycophenolate sodium mixed with silicon dioxide, lactose and magnesium stearate is exemplified.

U.S. Pat. No. 6,172,107 describes a pharmaceutical composition comprising a mycophenolate salt, the composition being adopted to release mycophenolate in the upper part of the intestinal tract. The delayed release of the active ingredient is provided by compositions having an enteric coating.

U.S. Pat. No. 6,306,900 describes a pharmaceutical composition comprising a mycophenolate salt, the composition being adopted to release mycophenolate in the upper part of the intestinal tract. The delayed release of the active ingredient is provided by compositions having an enteric coating. The supporting examples are enteric coated capsules.

U.S. patent application No. 2005/0013859 describes a solid dosage form, e.g. a tablet, comprising mycophenolic acid or mycophenolate salt and a process for its production. US 2005/0013859 describes both accelerated and delayed release compositions. Tablets comprising an enteric coating and a pharmacologically effective amount of mycophenolic acid or mycophenolate salt, wherein the mycophenolic acid or mycophenolate salt is present in an amount of from about 20% to about 95% by weight based on the total weight of the tablet including the enteric coating are exemplified.

WO 2007/093346 describes the use of mycophenolic acid in immunosuppression, particularly for prevention or treatment of transplant rejection and immuno-mediated and/or inflammatory diseases, wherein mycophenolic acid, the salt or the prodrug thereof is administered with an initial intensified dosage regimen. In particular, WO 2007/093346 describes the use of an enteric coated composition containing mycophenolate, or a salt or a prodrug thereof in the manufacture of a medication, whereby the dosage of mycophenolate during the first week to the first three months of treatment is up to about 3-fold the standard dosage of mycophenolate, and then mycophenolate is administered at standard daily dosage of mycophenolate.

WO 2009/047799 describes high dose solid unit oral pharmaceutical dosage form compositions comprising mycophenolate sodium as active agent in an amount of from greater than 720 mg to about 1500 mg and one or more pharmaceutically acceptable excipients(s). Particularly the dosage forms are meant for once a day or twice a day administration and provide the active agent in extended release form which is released in a sustained manner in-vivo for a prolonged duration.

Mycophenolate sodium is commercially available as enteric coated delayed-release tablets sold by Novartis under the trade name Myfortic®. Myfortic® delayed-release tablets are an enteric formulation of mycophenolate sodium that deliver the active moiety mycophenolic acid. Myfortic® is available for oral use as delayed-release tablets containing either 180 mg or 360 mg of mycophenolate sodium.

A derivative of mycophenolate, mycophenolate mofetil, has been developed and is commercially available in the United States under the trade name CellCept®.

Attempts in the prior art to prevent dissolution of mycophenolate compositions in the stomach, and provide release of mycophenolate in the upper part of the intestinal tract, have mainly employed an enteric coating. The application of an enteric coating to pharmaceutical compositions involves a relatively long coating process and the use of organic solvents which can both be expensive and impact the environment.

It is an object of the present invention to provide a pharmaceutical composition containing an active pharmaceutical ingredient selected from mycophenolic acid, a pharmaceutically acceptable salt thereof or combinations thereof, that prevents release of the active ingredient in the stomach and provides release in the upper tract of the intestines.

### SUMMARY OF THE INVENTION

In one embodiment there is provided a delayed release pharmaceutical composition comprising an active pharmaceutical ingredient selected from mycophenolic acid, a pharmaceutically acceptable salt thereof or combinations thereof, wherein said delayed release is matrix controlled. Preferably, the pharmaceutical composition comprises a matrix, wherein the matrix comprises an active pharmaceutical ingredient, and an enteric polymer. Preferably the active pharmaceutical ingredient is selected from mycophenolic acid, a pharmaceutically acceptable salt thereof or combinations thereof, such as mycophenolate sodium or mycophenolate mofetyl. Preferably, the pharmaceutical composition is in the form of a tablet. The enteric polymer is preferably selected from the group consisting of acrylic resins such as methacrylate copolymers, hydroxypropylmethyl cellulose phthalate, hydroxypropylmethyl cellulose acetate succinate and polyvinylacetate phthalate. The pharmaceutical composition may comprise an active pharmaceutical ingredient, an enteric polymer, and a disintegrant.

In another embodiment there is provided a process for preparing a delayed release pharmaceutical composition comprising combining an active pharmaceutical ingredient and an enteric polymer to provide a pharmaceutical composition matrix.

In yet another embodiment there is provided a process for preparing a delayed release pharmaceutical composition comprising an active pharmaceutical ingredient in the form of a tablet, the process comprising:
(i) granulating an active pharmaceutical ingredient, which is preferably mycophenolate sodium, with an enteric polymer and optionally one or more pharmaceutical acceptable excipients;
(ii) admixing the granules obtained from step (i) with one or more further pharmaceutical excipients; and
(iii) compressing the mixture obtained from step (ii) into tablets.

In another embodiment there is provided a process for preparing a delayed release pharmaceutical composition comprising an active pharmaceutical ingredient in the form of a tablet, the process comprising:
(a) mixing an active pharmaceutical ingredient, which is preferably mycophenolate sodium, with a diluent, which is preferably lactose monohydrate, and/or a suspending agent, which is preferably silicon dioxide, and optionally adding a disintegrant, which is preferably sodium starch glycolate;
(b) preparing a granulation solution comprising an enteric polymer, which is preferably hydroxypropylmethyl cellulose phthalate or polyvinyl acetate phthalate, and a solvent, which is preferably a mixture of ethanol and acetone;
(c) wet granulating the mixture obtained from step (i) using the granulation solution from step (ii), preferably drying the resulting granules;
(d) mixing the granules from step (c) with a disintegrant, which is preferably sodium starch glycolate, and/or a lubricant, which is preferably magnesium stearate;
(e) compressing the mixture from step (d) into tablets.

### DETAILED DESCRIPTION OF THE INVENTION

Delayed release in the context of the present invention means that the composition releases the active pharmaceutical ingredient(s) after passing through the stomach. In particular, the delayed release compositions of the present invention release the active pharmaceutical ingredient(s) in the upper tract of the intestine. Preferably, no dissolution of the composition occurs in the stomach. Such a dissolution profile can be tested in vitro using a dissolution test detailed in the Examples section below. For example, the compositions of the invention do not disintegrate for at least two hours when tested in artificial gastric juices such as 0.1N HCl of pH 1, and thereafter releases a substantial portion e.g. at least 60%, preferably more than 75%, of the labeled amount of active pharmaceutical ingredient(s) within 60 minutes in artificial intestinal juices such as phosphate buffer at pH 6.8.

In one aspect, the present invention provides a delayed release pharmaceutical composition comprising an active pharmaceutical ingredient, wherein said delayed release is matrix controlled (rather than coating controlled). Preferably, the pharmaceutical composition comprising a matrix, wherein the matrix comprises mycophenolic acid or a pharmaceutically acceptable salt thereof, and an enteric polymer.

The pharmaceutical composition is a delayed release composition. In the context of the present invention, the delayed release compositions do not release the active ingredient in the stomach but release of the active ingredient occurs distal to the stomach, preferably, in the intestines (usually the upper tract of the intestines). Preferably, not more than 5% of the active ingredient contained therein occurs in the stomach. Preferably, release of the active ingredient in the intestines is immediate, or within one hour. This delayed release profile is achieved by designing the composition so that it does not dissolve/disintegrate in the stomach but does dissolve/disintegrate in the intestines. The stomach is an acidic environment (approximately pH ˜3) whereas the intestines are a more basic environment (approximately pH ˜5.5 and above). The conditions of the stomach/intestines can therefore be replicated in vitro. For example, the pharmaceutical compositions of the invention do not release the active pharmaceutical ingredient for at least two hours in artificial gastric juices such as HCl of pH 1, and thereafter release a substantial portion e.g. at least 60%, preferably more than 75%, of active pharmaceutical ingredient within 60 minutes in artificial intestinal juices such as phosphate buffer at pH 6.8.

The active pharmaceutical ingredient, in the context of the present invention, is selected from the list comprising mycophenolic acid, a pharmaceutically acceptable salt thereof and combinations thereof, such as mycophenolate sodium or mycophenolate mofetyl. Most preferably, the active pharmaceutical ingredient is mycophenolate sodium.

Preferably, the active pharmaceutical ingredient is present in an amount greater than about 50%, more preferably between about 55% and about 99%, more preferably between about 60% and about 75%, most preferably between about 64% and about 71%, by weight (w/w) of the pharmaceutical composition.

The pharmaceutical compositions of the invention are preferably in the form of a tablet. Tablets of the present invention preferably exclude an enteric coating. This does not preclude any other type of coating being used however in a preferred embodiment, the tablets of the present invention exclude a coating (of any type), i.e. the pharmaceutical composition does not comprise a coating.

The pharmaceutical compositions can be prepared using any conventional process. In particular, a matrix comprised of granulated pharmaceutical compositions is preferred. The compositions can be prepared by conventional granulation techniques such as wet granulation. For example, the compositions can be prepared by wet granulation techniques applying high shear granulators. Accordingly, wet granulated compositions are preferred.

In a granulated composition of the invention, the matrix comprises an intra-granular portion and an extra-granular portion.

The enteric polymer employed in the present invention controls the location in the digestive system where the pharmaceutical composition dissolves and/or disintegrates. In particular, the enteric polymer is not affected by the acidic environment observed in the stomach area. Suitable enteric polymers include acrylic resins such as methacrylate copolymers, hydroxypropylmethyl cellulose phthalate, hydroxypropylmethyl cellulose acetate succinate, polyvinylacetate phthalate and combinations thereof. For example, the methacrylate copolymers may be methacrylic acid-methyl methacrylate (1:1) copolymers which are commercially available from Röhm GmbH & Co. KG in the “Eudragit L series of polymers” which dissolve at pH 5-6. Alternatively, the enteric polymer may be methacrylic acid-methyl methacrylate (1:2) copolymers which are commercially available from Röhm GmbH & Co. KG in the “Eudragit S series of polymers” which dissolve at pH greater than 6. Also, combinations of methacrylic acid-methyl methacrylate (1:1) copolymers and methacrylic acid-methyl methacrylate (1:2) are useful in the present invention.

In any embodiment of the present invention, the most preferred enteric polymers are hydroxypropylmethyl cellulose phthalate and polyvinyl acetate phthalate.

Preferably, the amount of enteric polymer is less than about 50%, more preferably between about 0.5% and about 30%, more preferably between about 1% and about 20%, more preferably between about 5% and about 15%, most preferably between about 8% and about 10% by weight (w/w) of the pharmaceutical composition.

The compositions of the present invention may also comprise one or more further pharmaceutically acceptable excipients selected from, but not limited to, disintegrants, binders, diluents, lubricants, glidants, suspending agents, plasticizers, emulsifying agents, sweetening agents, flavouring agents, and pigments.

Disintegrants are particularly preferred in any composition of the present invention. Disintegrants assist the break up of the composition in the intestine. Suitable disintegrants include, but are not limited to, alginic acid, calcium phosphate (tribasic), carboxymethylcellulose calcium, carboxymethylcellulose sodium, powdered cellulose, chitosan, colloidal silicon dioxide, croscarmellose sodium (crosslinked carboxymethyl cellulose sodium), crospovidone, docusate sodium, guar gum, hydroxypropyl cellulose, low-substituted hydroxypropyl cellulose, magnesium aluminium silicate, methylcellulose, microcrystalline cellulose, sodium alginate, sodium starch glycolate, starch, pregelatinized starch, and combinations thereof. Preferred disintegrants in any embodiment of the present invention are crospovidone, croscarmellose sodium, and sodium starch glycolate. The most preferred is sodium starch glycolate. Preferably, in any embodiment of the present invention, the disintegrant is present in an amount of about 1% to about 8%, more preferably about 3% to about 6.5%, more preferably about 4% to about 6%, most preferably about 4% by weight (w/w) of the pharmaceutical composition.

In a granulated pharmaceutical composition of the present invention, the disintegrant may be an intra-granular excipient, an extra-granular excipient or a combination of both intra-granular and extra-granular excipients. As suggested by the term itself, an intra-granular excipient is an excipient that is part of the granulated composition, i.e. the excipient is incorporated at the granulation phase of the process preparing a pharmaceutical composition. Likewise, an extra-granular excipient refers to an excipient that is not included in the granulate used to prepare the pharmaceutical composition but is added in the process preparing the pharmaceutical composition to the granulate, after its formation, in a subsequent process step. As an intra-granular excipient, a disintegrant is present in an amount of about 1% to about 6%, more preferably about 3% to about 5%, most preferably about 4% by weight (w/w) of the pharmaceutical composition. As an extra-granular excipient, a disintegrant is present in an amount greater than about 1%, preferably of about 1% to about 6%, more preferably about 3% to about 5%, most preferably about 3.5% to about 4.5% by weight (w/w) of the pharmaceutical composition. In one embodiment of the present invention, a disintegrant is included as both an intra-granular excipient and an extra-granular excipient. In such an embodiment, a disintegrant is present as an intra-granular excipient in an amount of about 1% to about 6%, more preferably about 3% to about 5%, most preferably about 4% by weight (w/w) of the pharmaceutical composition, and a disintegrant as an extra-granular excipient in an amount of about 1% to about 3%, more preferably about 2% by weight (w/w) of the pharmaceutical composition.

Suitable binders include, but are not limited to, acacia, alginic acid, carbomer copolymer, carbomer interpolymer, copovidone, microcrystalline cellulose, dextrin, ethylcellulose, gelatin, glucose (liquid), guar gum, hydroxypropyl cellulose, maltose, methylcellulose, polyethylene oxide, polyvinylpyrrolidone, povidone, starch, sodium carboxymethylcellulose and combinations thereof.

Suitable diluents used in the composition include, but are not limited to, calcium carbonate, calcium phosphate (dibasic and/or tribasic), calcium sulfate, microcrystalline cellulose, dextrates, dextrin, dextrose excipient, daolin, lactitol, lactose (anhydrous and/or monohydrate), maltose, mannitol, microcrystalline cellulose, sorbitol, starch, titanium dioxide, sucrose and combinations thereof. Preferably, the diluent is lactose, starch or titanium dioxide. Preferably, in any embodiment of the present invention, the diluent is present in an amount of about 1% to about 50%, more preferably about 1% to about 20%, more preferably about 5% to about 15%, more preferably about 10% to about 12% by weight (w/w) of the pharmaceutical composition.

In a granulated composition of the invention, a diluent may be included either as an intra-granular excipient or as an extra-granular excipient. As an intra-granular excipient, the diluent is preferably lactose monohydrate. As an extra-granular excipient, the diluent is preferably starch.

Suitable lubricants which can optionally be used in the composition include, but are not limited to sodium lauryl sulfate, calcium stearate, glyceryl behenate, magnesium stearate, polyethylene glycol, sodium stearyl fumarate, stearic acid, talc, or zinc stearate. Preferred lubricants include sodium lauryl sulfate, magnesium stearate or stearic acid. The most preferred lubricant is magnesium stearate. Preferably, in any embodiment of the present invention, the lubricant is present in an amount of about 0.5% to about 5%, more preferably about 0.5% to about 3% by weight (w/w) of the compositions.

Suitable glidants used in the compositions include, but are not limited to, talc, kaolin, glycerol monostearate, silicic acid, magnesium stearate, titanium dioxide and combinations thereof. Preferably, in any embodiment of the present invention, the glidant is present in an amount of between about 0% and about 5%.

Suitable suspending agents used in the compositions include, but are not limited to, colloidal silicon dioxide. Preferably, in any embodiment of the present invention, the suspending agent is present in an amount of about 1% to about 10%, more preferably about 1.5% to about 5%, most preferably about 2% to about 3% by weight (w/w) of the pharmaceutical composition.

Suitable plasticizers used in the compositions include, but are not limited to, triethyl citrate, triacetin, dibutyl sebacate, diethyl phthalate, and combinations thereof. Preferably, in any embodiment of the present invention, the plasticizer is present in an amount of about 0% to about 5%, more preferably about 0.5% to about 1.5%, by weight (w/w) of the pharmaceutical composition.

Suitable emulsifying agents used in the compositions include, but are not limited to, stearic acid, oleic acid, palmitic acid, esters of glycerol and fatty acids such as glycerol monostearate, acacia, gums such as guar gums, lecithin and combinations thereof. Preferably, in any embodiment of the present invention, the emulsifying agent is present in an amount of about 0% to about 5%, more preferably about 0.5% to about 1.5%, by weight (w/w) of the pharmaceutical composition.

Optionally, suitable sweetening agents, flavouring agents, pigments or combination thereof used in the compositions.

In any embodiment of the invention, the composition preferably comprises mycophenolate sodium and hydroxypropylmethyl cellulose phthalate (enteric polymer) or mycophenolate sodium and polyvinyl acetate phthalate (enteric polymer).

In an embodiment of the invention, the composition preferably comprises mycophenolate sodium, hydroxypropylmethyl cellulose phthalate (enteric polymer), and sodium starch glycolate (disintegrant).

In a further aspect of the present invention is provided a process for preparing any pharmaceutical composition of the invention described above. Preferably, the process of the present invention comprises combining an active pharmaceutical ingredient and an enteric polymer to provide a pharmaceutical composition matrix.

The pharmaceutical compositions of the present invention can be prepared by any conventional technique. For example, the compositions can be prepared using techniques such as direct compression, wet granulation, and dry granulation. The enteric polymers maybe incorporated as dry powders or through concentrated solutions in organic media. In this respect, the compositions can be prepared using granulation techniques. Preferably, the compositions are prepared using wet granulation.

In one embodiment of the invention in which the pharmaceutical composition is a tablet the invention provides a process for preparing a pharmaceutical composition of the invention comprising:
(i) granulating an active pharmaceutical ingredient with an enteric polymer and optionally one or more pharmaceutical acceptable excipients;
(ii) admixing the granules obtained from step (i) with one or more further pharmaceutical excipients; and
(iii) compressing the mixture obtained from step (ii) into tablets.

Suitable excipients useful in step (i) include, but are not limited to, diluents and/or suspending agents (as described above).

In step (i), a mixture of the active pharmaceutical ingredient and one or more pharmaceutical acceptable excipients may be prepared before the enteric polymer is added.

Preferably, step (i) involves wet granulating an active pharmaceutical ingredient with an enteric polymer and optionally one or more pharmaceutical acceptable excipients.

In a wet granulation process of the invention, any granulation solution can be employed. Preferably, the granulation solution is organic, more preferably the organic granulation solution comprises ethanol and/or acetone, most preferably the granulation solution is ethanol (95%) and/or acetone. In addition, the granulation solution preferably contains the enteric polymer. The enteric polymer is therefore introduced into the pharmaceutical composition via the granulation solution.

When using a granulation solution in step (i), the solution is preferably added gradually, for example added over a period of 2-3 minutes.

When step (i) above is a wet granulation process, the resulting wet mass obtained from the granulation process is preferably dried before the granules are mixed in step (ii). More preferably, the resulting wet mass is dried until the moisture content is less than about 2% by weight. Additionally or alternatively, the resulting granules may be sieved prior to step (ii) in order to provide particles in a selected size range. For example, the particle size can be selected so that 80-100%, preferably 90-100%, more preferably 100% of the granules have a particle size less than about 800 microns.

In step (ii), the mixture is preferably compressed into tablets having a hardness of 10 SCU (Strong Cobbs Unit) or more, preferably 15 SCU or more, more preferably 20 SCU or more, most preferably about 20 SCU.

In one embodiment of the present invention is provided a process for preparing a pharmaceutical composition in the form of tablet comprising:
(a) mixing an active pharmaceutical ingredient, which is preferably mycophenolate sodium, with a diluent, which is preferably lactose monohydrate, and/or a suspending agent, which is preferably silicon dioxide, and optionally adding a disintegrant, which is preferably sodium starch glycolate;
(b) preparing a granulation solution comprising an enteric polymer, which is preferably hydroxypropylmethyl cellulose phthalate or polyvinyl acetate phthalate, and a solvent, which is preferably a mixture of ethanol and acetone;
(c) wet granulating the mixture obtained from step (i) using the granulation solution from step (ii), preferably drying the resulting granules;
(d) mixing the granules from step (c) with a disintegrant, which is preferably sodium starch glycolate, and/or a lubricant, which is preferably magnesium stearate;
(e) compressing the mixture from step (d) into tablets.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Wet Granulation; HPMCP as Enteric Polymer with Ex-Granular Disintegrant

Mycophenolate Sodium, Silicon dioxide and Lactose anhydrous were mixed into a blend with a high shear mixer for one minute. The blend was further granulated with gradual addition of enteric polymer containing granulation solution for a few minutes. The granulation solution was prepared by mixing hydroxypropylmethyl cellulose phthalate with a solvent mixture of ethanol and acetone. The resulting wet mass was dried in a Fluid Bed Dryer at a nominal temperature of 50° C. till the moisture content was less than about 2%, and sieved to provide particles in a selected size range. The resulting granules (90% below 710 μm) were mixed with sodium starch glycolate and magnesium stearate and compressed into oval tablets having a hardness of about 20 SCU (Strong Cobbs Unit). Table 1 summarizes the composition of the tablets of Example 1.

TABLE 1
Formulation of Tablets of Example 1 by weight [mg]

| | | |
|---|---|---|
| Component | mg/tab | Function |

Intra-granular

| | | |
|---|---|---|
| Mycophenolate Sodium | 384.7 | API |
| Silicon Dioxide | 13.3 | Suspending agent |
| Lactose Anhydrous | 65.0 | Diluent |

Granulation Solution

| | | |
|---|---|---|
| Hydroxypropylmethyl | 52.6 | Enteric polymer |
| cellulose phthalate (HP-55) | | |
| Ethanol 95%* | | Granulation |
| Acetone* | | liquid/solvent |

Ex-granular

| | | |
|---|---|---|
| Sodium Starch Glycolate | 22.0 | Disintegrant |
| Magnesium Stearate | 6.4 | Lubricant |
| Total weight | 544.0 | |

### Example 2

### Wet Granulation; HPMCP as Enteric Polymer with Both Ex- and Intra-Granular Disintegrant

Mycophenolate Sodium, Silicon dioxide, Lactose anhydrous and sodium starch glycolate were mixed into a blend with a high shear mixer for one minute. The blend was further granulated with gradual addition of enteric polymer containing granulation solution for a few minutes. The granulation solution was prepared by mixing hydroxypropylmethyl cellulose phthalate with a solvent mixture of ethanol and acetone. The resulting wet mass was dried in a Fluid Bed Dryer at a nominal temperature of 50° C. till the moisture content was less than about 2%, and sieved to provide particles in a selected size range. The resulting granules (100% below 800 μm) were mixed with sodium starch glycolate and magnesium stearate and compressed into oval tablets having a hardness of about 20 SCU. Table 2 summarizes the composition of the tablets of Example 2.

TABLE 2
Formulation of Tablets of Example 2 by weight [mg]

| | | |
|---|---|---|
| Component | mg/tab | Function |

Intra-granular

| | | |
|---|---|---|
| Mycophenolate Sodium | 384.7 | API |
| Silicon Dioxide | 13.3 | Suspending agent |
| Lactose Anhydrous | 65.0 | Diluent |
| Sodium Starch Glycolate | 22.0 | Disintegrant |

Granulation Solution

| | | |
|---|---|---|
| Hydroxypropylmethyl | 52.6 | Enteric polymer |
| cellulose phthalate (HP-55) | | |
| Ethanol 95% | | |
| Acetone | | |

Ex-granular

| | | |
|---|---|---|
| Sodium Starch Glycolate | 12.0 | Disintegrant |
| Magnesium Stearate | 6.4 | Lubricant |
| Total weight | 556.0 | |

### Example 3

### Wet Granulation; PVAP as Enteric Polymer

Mycophenolate Sodium, Silicon dioxide and Lactose monohydrate were mixed into a blend with a high shear mixer for one minute. The blend is further granulated with gradual addition of enteric polymer containing granulation solution for a few minutes. The granulation solution was prepared by mixing commercial enteric formulation based on polyvinyl acetate phthalate (Opadry Enteric®) with a solvent mixture of ethanol and acetone. The resulting wet mass was dried in a Fluid Bed Dryer at a nominal temperature of 50° C. till the solvent content is less than about 2%, and sieved to provide particles in a selected size range. The resulting granules (90% below 600 μm) were mixed with starch and magnesium stearate and compressed into oval tablets having a hardness of about 21 SCU. Table 3 summarizes the composition of the tablets of Example 3.

TABLE 3
Formulation of Tablets of Example 3 by weight [mg].

| | | |
|---|---|---|
| Component | mg/tab | Function |

Intra-granular

| | | |
|---|---|---|
| Mycophenolate Sodium | 384.7 | API |
| Silicon Dioxide | 13.3 | Suspending agent |
| Lactose Monohydrate | 65.0 | Diluent |

Granulation Solution

| | | |
|---|---|---|
| Poly vinyl acetate Phtalate* | 50.9 | Enteric polymer |
| Titanium dioxide* | 18.0 | Diluent |
| Triethyl citrate* | 5.1 | Plasticizer |
| Stearic acid* | 5.1 | Emulsifying agent |
| FD&C yellow aluminum lake* | 2.6 | Pigment |
| Iron oxide yellow* | 0.5 | Pigment |
| Ethanol 95% | | Solvent |
| Acetone | | |

Ex-granular

| | | |
|---|---|---|
| Starch | 23.3 | Disintegrant |
| Magnesium Stearate | 6.5 | Lubricant |
| Total weight | 575.0 | |

*Component of Opadry enteric orange ® produced by Colorcon

### Example 4

### Wet Granulation; HPMCP as Enteric Polymer

Mycophenolate Sodium, Silicon dioxide and Lactose anhydrous were mixed into a blend with a high shear mixer for one minute. The blend was further granulated with gradual addition of enteric polymer containing granulation solution for a few minutes. The granulation solution was prepared by mixing hydroxypropylmethyl cellulose phthalate with a solvent mixture of ethanol and acetone. The resulting wet mass was dried in a Fluid Bed Dryer at a nominal temperature of 50° C. till the moisture content was less than about 2%, and sieved to provide particles in a selected size range. The resulting granules (90% below 710 μm) were mixed with starch and magnesium stearate and compressed into oval tablets having a hardness of about 15 SCU.

TABLE 4
Formulation of Tablets of Example 4 by weight [mg].

| | | |
|---|---|---|
| Component | mg/tab | Function |

Core

| | | |
|---|---|---|
| Mycophenolate Sodium | 384.7 | API |
| Silicon Dioxide | 13.3 | Suspending Agent |
| Lactose Anhydrous | 65.0 | Diluent |

Granulation Solution

| | | |
|---|---|---|
| Hydroxypropylmethyl cellulose | 69.0 | Enteric Polymer |
| phthalate (HP-55) | | |
| Ethanol 95% | | Granulating |
| Acetone | | Liquid/Solvent |

Ex-granular

| | | |
|---|---|---|
| Starch | 21.5 | Disintegrant |
| Magnesium Stearate | 6.5 | Lubricant |
| Total weight | 560.0 | |

### Example 5

### Dry Granulation; HPMCP as Enteric Polymer

Mycophenolate Sodium, Silicon dioxide, Lactose anhydrous, hydroxypropylmethyl cellulose phthalate and magnesium stearate are mixed into a blend with a high shear mixer for three minutes. The blend is then compacted applying a slugging tooling equipped tablet press machine or, alternatively, by roller compaction followed by low shear milling to obtain a uniformly sized granulate. This milled granulate is then mixed with sodium starch glycolate and magnesium stearate and compressed into oval tablets applying a tablet press machine.

TABLE 5
Formulation of Tablets of Example 5 by weight [mg].

| | | |
|---|---|---|
| Component | mg/tab | Function |

Dry granulation

| | | |
|---|---|---|
| Mycophenolate Sodium | 384.7 | API |
| Silicon Dioxide | 13.3 | Suspending Agent, |
| | | Glidant |
| Lactose Anhydrous | 65.0 | Diluent |
| Hydroxypropylmethyl cellulose | 70.0 | Enteric Polymer |
| phthalate (HP-55) | | |
| Magnesium Stearate | 3.5 | Lubricant |

Ex-granular

| | | |
|---|---|---|
| Sodium Starch Glycolate | 20.0 | Disintegrant |
| Magnesium Stearate | 3.5 | Lubricant |
| Total weight | 560.0 | |

### Example 6

### Dissolution Test

Tablets of the formulations of Examples 1-4 were dissolved for 120 min in acid medium followed by 60 min in buffer conditions. The acid medium was composed of 750 ml 0.1 N HCl at 37° C. and 100 RPM, whereas the buffer was 1000 ml Phosphate buffer at pH 6.8, 37° C. and 100 RPM (concentrated phosphate buffer was added to acid stage medium). The drug release profile was measured at the end of the acid stage (120 min) and after 130, 135, 140, 165 and 180 min in the buffer stage. Table 6 summarizes the dissolution data for Examples 1-4, their details are provided in Tables 1-4.

TABLE 6
Dissolution Profile of Examples 1-4

| Time | Example | Example | Example | Example |
|---|---|---|---|---|
| (Min) | 1 | 2 | 3 | 4 |
| 0 | 0% | 0% | 0% | 0% |
| 120 | 1% | 0% | 0% | 0% |
| 130 | 9% | 15% | 8% | 10% |
| 135 | 22% | 24% | 15% | 15% |
| 140 | 34% | 31% | 22% | 21% |
| 150 | 51% | 44% | 34% | 32% |
| 165 | 66% | 61% | 48% | 48% |
| 180 | 78% | 75% | 60% | 62% |

The results presented show the compositions of the invention exhibit a dissolution profile in which no, or virtually no, dissolution of the compositions occurs at low pH (i.e. simulating conditions in the stomach) and in which rapid dissolution of the compositions occurs at higher pH (i.e. simulating conditions in the upper tract of the intestines).

All matrix formulated samples were characterized by a low variability dissolution profile (RSD up to 32% at 135 min dissolution points for tests containing 3 tablets per batch). This is advantageous over enteric coated tablets for which the dissolution variability measured for 6 tablets was significantly higher (RSD of 50-90% at 135 min).

1. A delayed release pharmaceutical composition comprising an active pharmaceutical ingredient selected from mycophenolic acid, a pharmaceutically acceptable salt thereof or combinations thereof, wherein said delayed release is matrix controlled.

2. The pharmaceutical composition of claim 1, wherein the pharmaceutical composition comprises a matrix, wherein the matrix comprises an active pharmaceutical ingredient and an enteric polymer.

3. The pharmaceutical composition of claim 1, wherein release of the active pharmaceutical ingredient occurs in the intestines.

4. The pharmaceutical composition of claim 3, wherein release of the active pharmaceutical ingredient in the intestines is immediate, or within one hour.

5. The pharmaceutical composition of claim 1, wherein the pharmaceutical composition does not release the active pharmaceutical ingredient for at least two hours when tested in artificial gastric juices.

6. The pharmaceutical composition of claim 5, wherein the pharmaceutical composition thereafter releases at least 60% of active pharmaceutical ingredient within 60 minutes when tested in artificial intestinal juices.

7. The pharmaceutical composition of claim 1, wherein the active pharmaceutical ingredient is mycophenolate sodium.

8. The pharmaceutical composition of claim 1, wherein the active pharmaceutical ingredient is present in an amount greater than about 50% by weight (w/w) of the pharmaceutical composition.

9. The pharmaceutical composition of claim 1 in the form of a tablet.

10. The pharmaceutical composition of claim 9, wherein the tablet excludes an enteric coating.

11. The pharmaceutical composition of claim 9, wherein the tablet excludes any coating.

12. The pharmaceutical composition of claim 1, wherein the pharmaceutical composition is a matrix comprised of a granulated pharmaceutical composition.

13. The pharmaceutical composition of claim 12, wherein the granulated pharmaceutical composition is a wet granulated composition.

14. The pharmaceutical composition of claim 12, wherein the matrix comprises an intra-granular portion and an extra-granular portion.

15. The pharmaceutical composition of claim 2, wherein the enteric polymer is selected from the group consisting of acrylic resins such as methacrylate copolymers, hydroxypropylmethyl cellulose phthalate, hydroxypropylmethyl cellulose acetate succinate and polyvinylacetate phthalate.

16. The pharmaceutical composition of claim 15, wherein the enteric polymer is hydroxypropylmethyl cellulose phthalate or polyvinyl acetate phthalate.

17. The pharmaceutical composition of claim 15, wherein the amount of enteric polymer is less than about 50% by weight (w/w) of the pharmaceutical composition.

18. The pharmaceutical composition of claim 1, wherein the composition further comprises one or more pharmaceutically acceptable excipients selected from the group consisting of disintegrants, binders, diluents, lubricants, glidants, suspending agents, plasticizers, emulsifying agents, sweetening agents, flavouring agents, and pigments.

19. The pharmaceutical composition of claim 18, wherein the composition comprises at least one disintegrant selected from the group consisting of alginic acid, calcium phosphate (tribasic), carboxymethylcellulose calcium, carboxymethylcellulose sodium, powdered cellulose, chitosan, colloidal silicon dioxide, croscarmellose sodium (crosslinked carboxymethyl cellulose sodium), crospovidone, docusate sodium, guar gum, hydroxypropyl cellulose, low-substituted hydroxypropyl cellulose, magnesium aluminium silicate, methylcellulose, microcrystalline cellulose, sodium alginate, sodium starch glycolate, starch, pregelatinized starch, and combinations thereof.

20. The pharmaceutical composition of claim 19, wherein the disintegrant is crospovidone, croscarmellose sodium, or sodium starch glycolate.

21. The pharmaceutical composition of claim 19, wherein the disintegrant is present in an amount of about 1% to about 8% by weight (w/w) of the pharmaceutical composition.

22. The pharmaceutical composition of claim 19, wherein the disintegrant is present as an intra-granular excipient, an extra-granular excipient or a combination of both intra-granular and extra-granular excipients.

23. The pharmaceutical composition of claim 22, wherein the disintegrant is present as an intra-granular excipient in an amount of about 1% to about 6% by weight (w/w) of the pharmaceutical composition.

24. The pharmaceutical composition of claim 22, wherein the disintegrant is present as an extra-granular excipient in an amount of about 1% to about 6% by weight (w/w) of the pharmaceutical composition.

25. The pharmaceutical composition of claim 22, wherein the disintegrant is included as both an intra-granular excipient and an extra-granular excipient.

26. The pharmaceutical composition of claim 25, wherein the disintegrant is present as an intra-granular excipient in an amount of about 1% to about 6% by weight (w/w) of the pharmaceutical composition, and as an extra-granular excipient in an amount of greater than about 1% by weight (w/w) of the pharmaceutical composition.

27. The pharmaceutical composition of claim 1, wherein the composition comprises mycophenolate sodium and hydroxypropylmethyl cellulose phthalate (enteric polymer) or mycophenolate sodium and polyvinyl acetate phthalate (enteric polymer).

28. The pharmaceutical composition of claim 1, wherein the composition comprises mycophenolate sodium, hydroxypropylmethyl cellulose phthalate (enteric polymer), and sodium starch glycolate (disintegrant).

29. A process for preparing a pharmaceutical composition as defined in claim 1 comprising combining mycophenolic acid, a pharmaceutically acceptable salt thereof or combinations thereof and an enteric polymer to provide a pharmaceutical composition matrix.

30. The process of claim 29, wherein the pharmaceutical composition is prepared by direct compression, wet granulation, or dry granulation.

31. The process of claim 29, wherein the enteric polymer is incorporated as a dry powder or through a solution.

32. The process of claim 31, wherein the solution is a concentrated solution in an organic media.

33. A process for preparing a pharmaceutical composition as defined in claim 1 in the form of a tablet comprising:
(iv) granulating an active pharmaceutical ingredient selected from mycophenolic acid, a pharmaceutically acceptable salt thereof or combinations thereof, with an enteric polymer and optionally one or more pharmaceutical acceptable excipients;
(v) admixing the granules obtained from step (i) with one or more further pharmaceutical excipients; and
(vi) compressing the mixture obtained from step (ii) into tablets.

34. The process of claim 33, wherein in step (i), a mixture of the active pharmaceutical ingredient and one or more pharmaceutical acceptable excipients are prepared before the enteric polymer is added.

35. The process of claim 33, wherein step (i) involves wet granulating an active pharmaceutical ingredient with an enteric polymer and optionally one or more pharmaceutical acceptable excipients.

36. The process of claim 33, wherein the process is a wet granulation process and the granulation solution is organic.

37. The process of claim 36, wherein the granulation solution comprises ethanol and/or acetone.

38. The process of claim 36, wherein the granulation solution contains the enteric polymer.

39. A process for preparing a pharmaceutical composition as defined in claim 1 in the form of a tablet, comprising:
(a) admixing an active pharmaceutical ingredient with a diluent and/or a suspending agent and optionally adding a disintegrant;
(b) preparing a granulation solution comprising an enteric polymer and a solvent;
(c) wet granulating the mixture obtained from step (i) using the granulation solution from step (ii);
(d) admixing the granules from step (c) with a disintegrant and/or a lubricant;
(e) compressing the mixture from step (d) into tablets.

40. The process of claim 39, wherein the active pharmaceutical ingredient is mycophenolate sodium, the diluent is lactose monohydrate, the suspending agent is silicon dioxide, the disintegrant is sodium starch glycolate, the enteric polymer is hydroxypropylmethyl cellulose phthalate or polyvinyl acetate phthalate, the solvent is a mixture of ethanol and acetone, and the lubricant is magnesium stearate, and wherein the resulting granules from step (c) are dried prior to admixing them with a disintegrant and/or lubricant in step (d).
